(19) United States
(12) Patent Application Publication
(10) Pub. No.: US 2026/0216054 A1
(43) Pub. Date: Jul. 30, 2026

(54) COMPOSITIONS COMPRISING EPILOBIUM FLEISCHERI EXTRACT FOR TREATING SIGNS OF AGING

(71) Applicant: DSM IP Assets B.V., Maastricht (NL)

(72) Inventor: Leithe BUDEL, Kaiseraugst (CH)

(21) Appl. No.: 19/146,835

(22) PCT Filed: Jan. 12, 2024
(86) PCT No.: PCT/EP2024/050739

§ 371 Date: Jul. 9, 2025

(30) Foreign Application Priority Data
Jan. 13, 2023 (EP) 23151606.3

(51) Int. Cl. A61K 8/9789; A61K 8/06; A61K 8/34; A61K 8/36; A61K 8/365; A61Q 19/08

(52) U.S. Cl. CPC A61K 8/9789; A61K 8/062; A61K 8/345; A61K 8/36; A61K 8/365; A61Q 19/08

(57) ABSTRACT

The present invention relates to the (cosmetic) use of an Epilobium fleischeri extract for treating the signs of skin aging.

The present invention relates to the (cosmetic) use of an Epilobium fleischeri extract for treating the signs of skin aging.

Life expectancy is constantly increasing in many countries around the globe. Accordingly, the demand for treatments counteracting or even reversing aging processes is constantly growing, not only for age-related diseases but also for dealing with the physical signs of aging. Thus, it is not surprising that the need for cosmetic products, that may be applied topically on skin to combat the signs of skin aging such as wrinkles and age spots, has been growing significantly lately.

Aging of the skin is a complex process. Intrinsic aging is an inevitable physiological process that results in thin, dry skin, fine wrinkles, decreased elasticity, aberrant pigmentation, hair greying, and hair loss. Extrinsic aging is caused by external environmental factors notably solar radiation and air pollution and results in coarse wrinkles, loss of elasticity, laxity, and rough-textured appearance. Other factors that can further contribute to an aged appearance include general poor health, an unhealthy diet, cigarette smoking, and alcohol.

Today, it is well established that cell senescence plays an important role in aging processes. Cell senescence is a physiological process and a tumour-suppressive cell fate characterized by a permanent and irreversible cell cycle arrest as well as acquisition of a pro-inflammatory and proteolytic secretome. Cells that become senescent grow in size and cease to replicate. In addition, senescent cells generate a potent mix of molecules known as the senescence-associated secretory phenotype (SASP), which provokes the immune system into an inflammatory state, disrupts tissue structure and function, and in turn encourages nearby cells to also become senescent. The SASP is one of the key characteristics that distinguish senescent cells from quiescent, terminally differentiated, and other types of non-proliferating cells.

Intrinsic aging is typically correlated with an increase of senescent cells, including senescent skin cells. Senescence of dermal fibroblasts for example is considered a key driver of the aging-related phenotype in skin. Hence, there is an ongoing need to find agents which are able to selectively get rid of these errant cells.

Senolytics, which are typically understood as agents able to selectively eliminate senescent cells, were the first potential senotherapy to be successfully tested in preclinical in vivo models. In other words, an agent is considered as having senolytic activity when its activity includes specifically inducing or promoting cell death, in particular apoptotic cell death, of senescent cells in the respective tissue. Several senolytic agents have been identified by now such as navitoclax. Said senolytic agents are ultimately able to selectively eliminate senescent cells by inducing apoptosis processes that do not occur in non-senescent or normal cells, keeping these therefore unaffected.

A drawback of currently known senolytic agents is that they are often of synthetic origin, have rather complex chemical structures that require significant efforts to be obtained, exhibit low solubility in conventional cosmetic oils and/or may have severe undesired side effects.

Therefore, there is an ongoing need for senolytic agents of natural origin with reduced undesired side effects and that are obtainable in an economic manner.

Surprisingly, it has now been found that Epilobium fleischeri extract exhibits a senolytic activity on human dermal fibroblasts and can thus be used to treat the signs of skin aging.

Accordingly, in a first embodiment, the present invention relates to the cosmetic use of an Epilobium fleischeri extract as an active ingredient for the prevention, reduction, or treatment of the signs of skin aging.

In another embodiment, the invention also provides the use of an Epilobium fleischeri extract as an active ingredient in, and for the manufacture of, topical compositions for treating signs of skin aging.

In a further embodiment, the present invention also provides an Epilobium fleischeri extract as an active ingredient for use in the selective elimination of senescent skin cells.

The invention also provides the use of an Epilobium fleischeri extract as an active ingredient in, and for the manufacture of, topical compositions for the selective elimination of senescent skin cells.

The invention also provides a cosmetic method for treating signs of skin aging, comprising topically applying to an external surface of the human body a topical composition containing an Epilobium fleischeri extract.

The present invention also refers to a cosmetic skin and scalp care method for selective removal of senescent cells in the skin preferably of senescent human dermal fibroblasts by applying, onto the skin or the scalp in need, at least an effective amount of an extract of an Epilobium fleischeri extract; or a composition comprising at least said extract.

As used herein, the term ‘cosmetic’ refers to a treatment which does not cure, treat, or prevent a disease or disorder, but instead serves as a skincare product intended to beautify or improve the appearance of the skin, e.g., the colour or texture of the skin.

In the context of this invention, the term ‘treating’ denotes reducing, preventing, or eliminating.

The expression ‘signs of skin aging’ as used herein denotes one or more characteristics of intrinsic or chronological aging such as thin skin, fine wrinkles, decreased elasticity and aberrant pigmentation. These signs affect everyone, whatever their skin type or state of health. As noted above, the process may be amplified by extrinsic factors such as exposure to sunlight, pollutants, and cigarette smoke.

Accordingly, the term ‘treating the signs of skin aging’ includes the prevention, reduction or treatment of fine lines, wrinkles, crow's feet, sagging, skin thinning, age spots as well as improving skin elasticity or skin firmness without being limited thereto.

The term ‘prevention, reducing or treating skin aging’ also encompass smoothening of wrinkles and fine lines as well as decreasing their volume and depth in a person in need thereof.

The term ‘external surface of the human body’ as used herein encompasses the skin as well as the scalp (including hair and axilla). Preferably, in all embodiments of the present invention the external surface of the human body treated according to the present invention is the face, neck and/or body skin, most preferably the face (including the (lateral) cheek, forehead, nose, chin).

The term ‘skin cells’ as used herein may refer to any skin resident cell type (such as dermal fibroblasts, melanocytes, hair follicle dermal papilla cells, and epidermal keratinocytes), or cell types from skin appendages (such as sebocytes), or a mixture thereof. Preferably in all embodiments of the present invention, the term refers to (human) dermal fibroblasts.

In the context of this invention, the term ‘senescent’ refers to a state of permanent cell cycle arrest in which cells remain metabolically active and adopt characteristic phenotypic changes. The establishment of this phenotype is believed to be either the result of telomere shortening after several cell divisions (replicative senescence) or a response to stress stimuli (stress-induced senescence). One of the defining features of senescent cells is their stable cell cycle arrest. This cell cycle exit is controlled by activation of the p53/p21 and p16^{INK4a}/Rb tumour suppressor pathways. Unlike quiescent cells, senescent cells are nonresponsive to mitogenic or growth factor stimuli; thus, they are unable to re-enter the cell cycle even in advantageous growth conditions. Senescent cells are also distinct from terminally differentiated cells, which are also irreversibly withdrawn from the cell cycle. While terminal differentiation is the result of a defined developmental programme, which turns undifferentiated precursors into specialized effector cells, senescence is mainly implemented as a cellular stress response.

Senescent cells can unambiguously be identified in vitro and in vivo since they exhibit a number of characteristics that allow their explicit identification.

For example, senescent cells often appear multinucleated, large and extended, and exhibit spindle and vacuolisation features. They also display modifications in the organisation of chromatin that can help identify them. In normal cells, DNA staining reveals completely uniform colour outlines, whereas senescent cells usually show dot-like patterns, known as senescence-associated heterochromatic foci (SAHF). This phenomenon is due to intensive remodelling in the chromatin, which results in less susceptibility for digestion by nucleases.

Furthermore, senescence-related chromatin remodelling leads to profound transcriptional changes. Among the assortment of upregulated genes is a prominent subset of genes that encode secreted proteins, including cytokines and chemokines with proinflammatory properties, as well as various growth factors and proteases that together alter tissue structure and function, collectively known as SASP. The SASP is one of the key characteristics that distinguish senescent cells from quiescent, terminally differentiated, and other types of non-proliferating cells.

A distinctive measurable feature of senescent cells is the presence of β-galactosidase enzymatic activity. This enzyme normally displays activity at pH 4.0 within lysosomes, but in senescent cells it is also active at pH 6.0. This phenomenon is termed senescence associated-β-galactosidase (SA-β-gal) activity and is thought to be due to an enlargement in the structure of lysosomes in senescent cells. SA-β-gal activity is detectable by histochemical staining by using X-gal as a substrate for SA-β-gal. Since SA-β-gal activity is detected in most senescent settings, both in vitro and in vivo, it is considered a de facto hallmark of senescence. SA-β-gal can be visualized in the cells to quantify the senescence levels using flow cytometry.

The term ‘senolytic activity’ as used herein refers to specifically (also: selectively) inducing or promoting cell death of senescent cells. This may also be considered as ability for eliminating selectively senescent cells, for example, by inducing or promoting apoptosis. It may also be the capability to selectively eliminate senescent cells found in a tissue with a reduced or even no harmful effect on the normal resident (i.e., non-senescent) cells of the said tissue. Such senolytic property may be beneficial, for example, for preventing or attenuating the increase of the number of age-related senescent cells and/or stress-related senescent cells within a tissue.

Selective elimination of senescent cells may be determined by the change in ratio of senescent (decreasing) versus proliferating cells (increasing) based on SA-β-gal activity in the cell population model after incubation with a fixed concentration of a test compound in a suitable medium as illustrated in the examples. Such a cell population model may contain a set percentage ratio of senescent versus proliferating cells. By comparing with and without added test compound conditions, senolytic activity can be evaluated.

Compounds may be determined as capable of selectively eliminating senescent cells if the calculated senescent cell ratio (based on SA-β-gal activity) is reduced in a statistically significant manner versus the control (no compound added during incubation).

Preferably, in one embodiment the (total) number of senescent cells is reduced by at least 20%, more preferably by at least 25%, most preferably by at least 30%, such as by at least 40%, based on the initial number of senescent cells present in the treated sample such as the skin tissue. It is well understood, that concomitantly, the number of normal cells is increased by the same factor.

Preferably, in another embodiment, the number of senescent cells is reduced by at least a statistical difference in percentage between the control and treated conditions, more preferably by at least 50% in difference (half of original senescent cells removed), most preferably by at least 90% in difference, based on the initial number of senescent cells present. It is well understood, that concomitantly, the number of normal cells is increased by the same factor.

As used herein, the term ‘extract’ may be understood in the broadest sense as generally understood in the art. An extract may be any substance made by extracting a part of a raw material (here: flowers, leafs, seeds and/or stems of Epilobium fleischeri).

In a preferred embodiment, the Epilobium fleischeri extract has senolytic activity including specifically inducing or promoting cell death of senescent cells in the tissue. In this context, cell death preferably is apoptotic cell death.

Accordingly, the Epilobium fleischeri extract, according to the present invention, may be obtained by any means known to a person skilled in the art. It may optionally be obtained by a commercial supplier or may be partly or completely prepared from an Epilobium fleischeri plant or one or more parts thereof.

The extract may be any kind of extract. It may be prepared from fresh flowers, leafs, seeds and/or stems of Epilobium fleischeri or may be prepared from the dried flowers, leafs, seeds and/or stems of Epilobium fleischeri or may be prepared from the frozen or freeze-dried flowers, leafs, seeds and/or stems of Epilobium fleischeri. Preferably, in all embodiments of the present invention, the extract is derived from the flowers and leafs or from the flowers, leafs and stems of Epilobium fleischeri. Preferably in all embodiments, the Epilobium fleischeri extract is derived from the flowers, leafs and stems.

Preferably, the flowers, leafs, seeds and/or stems of Epilobium fleischeri may be used in chopped, blended or pulverized form. In a preferred embodiment, the extract is prepared from the dried flowers, leafs and/or stems of Epilobium fleischeri, in particular from dried and pulverized flowers, leafs and/or stems of Epilobium fleischeri.

Typically, extraction is achieved by using a solvent. Accordingly, in a preferred embodiment, the extract according to the present invention is obtained from solvent extraction methods including cold or hot extraction, ultrasonic extraction, reflux cooling, needle extraction, and microwaves extraction. In a preferred embodiment, the extract is obtained by performing an ultrasonic extraction method.

One or more solvents may be used for extraction. Such solvent may be any solvent suitable for this purpose. Typically, a solvent is liquid under the conditions it is used for extraction. A solvent may be selected from the group consisting of an organic solvent (e.g., an alcohol (e.g., methanol, ethanol, propanol, butanol, pentanol, phenol, glycerol, 1,3-butylene glycol, propane diol, etc.), water (including hot water or subcritical water), aqueous buffer, and a combination of two or more thereof. An organic solvent may be aliphatic or aromatic. Preferably, the solvent may be hydrated.

Herein, a solvent may be added to the flowers, leafs, seeds and/or stems of Epilobium fleischeri and left together for a certain time. In a further step, the solid parts are preferably separated by the primary extract. This may be achieved by any means such as, e.g., filtration, sieving, ultrafiltration, crossflow filtration, centrifugation, precipitation over time, or a combination of two or more thereof.

Preferably, in all embodiments of the present invention the Epilobium fleischeri extract used according to the present invention is prepared by extraction with a polar solvent such as water, ethanol, glycerin, glycols or mixtures thereof, preferably with ethanol or water as well as mixtures thereof.

In all embodiments of the present invention, the flowers, leafs, seeds and/or stems of Epilobium fleischeri are preferably extracted with an ethanol/water mixture containing between 10 and 90% (m/m) ethanol or containing between 25 and 85% (m/m) ethanol, or containing between 40 and 75% (m/m) ethanol, or containing between 50 and 70% (m/m) ethanol, or containing between 50 and 60% (m/m) ethanol.

In all embodiments of the present invention, extraction is advantageously conducted at a temperature in the range of 10 to 150° C., or 15 to 100° C., or 20 to 80° C., or 20 to 70° C. or 20 to 60° C., or approximately 20° C. In a preferred embodiment, extraction is conducted at a temperature selected in the range of ambient temperature (i.e., 15 to 25° C., in particular approximately 20° C.) to 60° C.

In all embodiments of the present invention, extraction is preferably conducted at a temperature selected in the range of approximately 20° C. (RT) to 60° C. and ambient pressure (i.e., 980 to 1200 mbar) for 1 to 3 hours.

In all embodiments of the present invention, the Epilobium fleischeri extract can either be a liquid extract, such as an aqueous extract, or a dry extract.

As used herein, the term ‘dry extract’ may be understood in the broadest sense as an extract according to the present invention as dry matter, i.e., the extraction product without the solvent. The dry extract may optionally be physically present as dry matter. It will, however, be directly understood that, in calculations referring a percentage referred to dry matter, it does not have to be dry matter present in physical form, but the dry extract may also be present in dissolved form. Then, in the calculation, the mass of the solvent is mathematically subtracted from the total mass.

Dry matter (in physical form) may be obtained by a suitable extraction method, followed by a step of drying the extract obtained. The drying may be performed by any method suitable for this purpose known in the art. Drying means removing the one or more solvents used for extraction. Removing the one or more solvents may be performed by any means. For example, it may be achieved by subjecting the extract to a hot and dry atmosphere and/or placing the extract on a heated plate in order to evaporate the solvent. For example, drying may be achieved by evaporation in a vacuum and/or at elevated temperature (e.g., in a rotary (vacuum) evaporator) or by crystallization of the solid material of interest. Alternatively, or additionally, drying may be achieved by atomisation or by lyophilisation.

Preferably, in all embodiments of the present invention, the Epilobium fleischeri extract is an aqueous or a dry (in physical form) extract. An aqueous Epilobium fleischeri extract is preferably prepared by a process encompassing the steps of milling aerial parts such as in particular the flowers, leafs and stems of Epilobium fleischeri which preferably have preferably been dried beforehand, more preferably under hot air followed by extraction of the dried plants with a solvent, preferably with a mixture of ethanol/water with all the definitions and preferences as given herein, followed by removal of the ethanol e.g., by (vacuum) distillation to obtain the aqueous Epilobium fleischeri extract.

The dry extract is obtained by vacuum distillation of ethanol and water, preferably at a temperature selected in the range of 50 to 70° C. until obtainment of the dry extract.

In all embodiments of the present invention said aqueous or dry extract of Epilobium fleischeri may be admixed with further ingredients such as preferably water, glycerine, citric acid and preservative(s) such as potassium sorbate, e.g., before being used to be incorporated into a topical composition according to the present invention.

Preferably, in all embodiments of the inventions the Epilobium fleischeri extract to be used in the topical compositions as disclosed herein contains (i) from 5 to 10 wt.-% based on dry matter of the Epilobium fleischeri flower, leaf stem extract, (ii) from 25 to 50 wt.-% of water, (iii)>50 wt.-% of a diol such as preferably glycerine, and (iv) from 0.1 to 1 wt.-% of one or more additives such as preferably citric acid and/or potassium sorbate while advantageously, all ingredients sum up to 100 wt.-%.

In all embodiments of the present invention the Epilobium fleischeri extract is preferably administered in the form of a topical composition comprising an effective amount of the Epilobium fleischeri extract and a cosmetically acceptable carrier.

The term ‘an effective amount’ refers to an amount necessary to obtain the desired physiological effect. The physiological effect may be achieved by one application dose or by repeated applications. The dosage administered may, of course, vary depending upon known factors, such as the physiological characteristics of the particular composition comprising the Epilobium fleischeri extract and its mode and route of administration; the age, health and weight of the recipient; the kind of concurrent treatment; the frequency of treatment; and the effect desired and can be adjusted by a person skilled in the art.

The term ‘cosmetically acceptable carrier’ (also referred to herein as carrier) refers to all vehicles/carriers conventionally used in topical composition, i.e., which are suitable for topical application to the keratinous tissue, have good aesthetic properties, are compatible with the actives present in the composition, and will not cause any unreasonable safety or toxicity concerns. Such carriers are well-known to one of ordinary skill in the art and can include one or more compatible liquid(s) or solid filler diluent(s), excipient(s), additive(s) or vehicle(s) which are suitable for application to skin.

Generally, the amount of the Epilobium fleischeri extract in the topical compositions according to the present invention is selected in the range from 0.001 to 10 wt.-%, more preferably from 0.01 to 8 wt.-%, even more preferably from 0.1 to 7 wt.-%, furthermore preferably from 0.1 to 5 wt.-% and still more preferably from 0.1 to 3 wt.-%, based on the total weight of the topical composition. Further suitable ranges are from 0.05 to 2 wt.-% and from 0.1 to 1 wt.-%.

The exact amount of carrier will depend upon the actual level of the Epilobium fleischeri extract and of any other optional ingredients that one of ordinary skill in the art would classify as distinct from the carrier (e.g., other active ingredients)

In an advantageous embodiment, the topical compositions according to the present invention comprise from about 50% to about 99%, preferably from about 60% to about 98%, more preferably from about 70% to about 98%, such as in particular from about 80% to about 95% of a carrier, based on the total weight of the topical composition.

In a particular advantageous embodiment, the carrier consists furthermore of at least 40 wt.-%, more preferably of at least 50 wt.-%, most preferably of at least 55 wt.-% of water, such as in particular of about 55 to about 90 wt.-% of water.

The topical compositions according to the present invention are preferably prepared by admixing the Epilobium fleischeri extract with all the definitions and preferences as given herein with/into a cosmetically acceptable carrier.

The topical composition may comprise further ingredients, which may form part of the carrier. Such ingredients are particularly surfactants, emulsifiers, thickeners, and oils. Such suitable surfactants, emulsifiers, thickeners, and oils are well known to a person skilled in the art.

The topical compositions of the invention (including the carrier) may comprise further conventional (cosmetic) adjuvants and additives, such as preservatives/antioxidants, fatty substances/oils, water, organic solvents, silicones, thickeners, softeners, emulsifiers, antifoaming agents, aesthetic components such as fragrances, surfactants, fillers, anionic, cationic, non-ionic or amphoteric polymers or mixtures thereof, propellants, acidifying or basifying agents, dyes, colourings/colorants, abrasives, absorbents, chelating agents and/or sequestering agents, essential oils, skin sensates, astringents, pigments or any other ingredients usually formulated into such compositions.

In accordance with the present invention, the compositions according to the invention may also comprise further cosmetically active ingredients conventionally used in topical composition. Exemplary active ingredients encompass skin lightening agents; UV-filters, agents for the treatment of hyperpigmentation; agents for the prevention or reduction of inflammation; firming, moisturizing, soothing, and/or energizing agents as well as agents to improve elasticity and skin barrier.

If nothing else is stated, the excipients, additives, diluents, etc. mentioned in the following are suitable for the compositions according to the present invention. The necessary amounts of the cosmetic and dermatological adjuvants and additives can, based on the desired product, easily be determined by the skilled person.

The additional ingredients can either be added to the oily phase, the aqueous phase or separately as deemed appropriate. The mode of addition can easily be adapted by a person skilled in the art.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

The topical compositions according to the present invention are in particular skin care preparations, or functional (anti-aging) preparations.

Examples of skin care preparations are, in particular, light protective preparations (sun care preparations), anti-aging preparations, preparations for the treatment of photo-aging, body oils, body lotions, body gels, treatment creams, skin protection ointments, moisturizing preparations such as moisturizing gels or moisturizing sprays, face and/or body moisturizers, as well as skin lightening preparations.

Examples of functional preparations are topical composition containing active ingredients such as hormone preparations, vitamin preparations, vegetable extract preparations, anti-aging preparations, and/or antimicrobial (antibacterial or antifungal) preparations without being limited thereto.

The topical composition according to the present invention can be in a wide variety of forms. Non limiting examples include simple solutions (e.g. aqueous, organic solvent, or oil based), emulsion or micro emulsion (in particular of oil-in-water (O/W) or water-in-oil (W/O) type, silicone-in-water (Si/W) or water-in-silicone (W/Si) type, PIT-emulsion, multiple emulsion (e.g. of oil-in-water-in oil (O/WV/O) or water-in-oil-in-water (W/O/W) type) or pickering emulsions), as well as solid forms (e.g. hydrogels, alcoholic gels, lipogels, sticks, flowable solids, or amorphous materials).

These product forms may be used for a number of applications, including, but not limited to, gels, creams, ointments, lotions, serums, powder, aerosol sprays or two component dispensing systems.

In a preferred embodiment, the topical composition according to the present invention are emulsions and/or gels. Even more preferably, the topical composition are emulsions which contain an oily phase and an aqueous phase such as in particular O/W, W/O, Si/W, W/Si, O/V/O, W/O/W multiple or a pickering emulsions.

The amount of the oily phase (i.e. the phase containing all oils and fats including the polar oils) present in such emulsions such as in particular O/W, W/O, Si/W, W/Si, O/W/O, W/O/WV multiple or a pickering emulsions is preferably at least 10 wt.-%, such as in the range from 10 to 60 wt.-%, preferably in the range from 15 to 50 wt.-%, most preferably in the range from 15 to 40 wt.-%, based on the total weight of the composition.

The oil phase according to the invention preferably comprises oils selected from butylenglykoldicaprylat/-dicaprat, propylenglykoldicaprylat/-dicaprat, dicaprylylether, C12-15-Alkylbenzoat, C18 38 fatty acid triglyceride, dibutyladipate, cyclomethicone, dimethicone, 2-phenylethylbenzoat, isopropyl lauroyl sarkosinate, caprylic/capric triglyceride as well as mixtures thereof.

The amount of the aqueous phase present in such emulsions is preferably at least 20 wt.-%, such as in the range from 20 to 90 wt.-%, preferably in the range from 30 to 80 wt.-%, most preferably in the range from 30 to 70 wt.-%, based on the total weight of the composition.

Advantageously in all emulsions of the present invention the ratio of oily phase to aqueous phase is selected in the range of 40:60 to 30 to 70.

In one particular advantageous embodiment, the topical composition according to the present invention are in the form of an oil-in-water (O/W) emulsion comprising an oily phase dispersed in an aqueous phase in the presence of an O/W emulsifier. The preparation of such O/W emulsions is well known to a person skilled in the art.

If the cosmetic composition according to the invention is an O/W emulsion, then it contains advantageously at least one O/W- or Si/WV-emulsifier selected from the list of, glyceryl stearate citrate, glyceryl stearate SE (self-emulsifying), stearic acid, salts of stearic acid, polyglyceryl-3-methylglycosedistearate. Further suitable emulsifiers are phosphate esters and the salts thereof such as cetyl phosphate (e.g., as Amphisol® A from DSM Nutritional Products Ltd.), diethanolamine cetyl phosphate (e.g., as Amphisol® DEA from DSM Nutritional Products Ltd.), potassium cetyl phosphate (e.g., as Amphisol® K from DSM Nutritional Products Ltd.), sodium cetearylsulfate, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, cetearyl glucoside, lauryl glucoside, decyl glucoside, sodium stearoyl glutamate, sucrose polystearate and hydrated polyisobutene. Furthermore, one or more synthetic polymers may be used as an emulsifier.

For example, PVP eicosene copolymer, acrylates/C10-30 alkyl acrylate crosspolymer, and mixtures thereof.

The at least one O/W, respectively Si/W emulsifier is preferably used in an amount of 0.5 to 10 wt.-%, in particular in the range of 0.5 to 6 wt.-%, such as more in particular in the range of 0.5 to 5 wt.-%, such as most in particular in the range of 1 to 4 wt.-%, based on the total weight of the cosmetic composition.

Particular suitable O/W emulsifiers to be used in the topical composition according to the invention encompass phosphate ester emulsifiers such as advantageously 8-10 alkyl ethyl phosphate, C9-15 alkyl phosphate, ceteareth-2 phosphate, ceteareth-5 phosphate, ceteth-8 phosphate, ceteth-10 phosphate, cetyl phosphate, C6-10 pareth-4 phosphate, C12-15 pareth-2 phosphate, C12-15 pareth-3 phosphate, DEA-ceteareth-2 phosphate, DEA-cetyl phosphate, DEA-oleth-3 phosphate, potassium cetyl phosphate, deceth-4 phosphate, deceth-6 phosphate and trilaureth-4 phosphate.

A particular suitable O/W emulsifier to be used in the topical composition according to the invention is potassium cetyl phosphate e.g., commercially available as Amphisol® K at DSM Nutritional Products Ltd Kaiseraugst.

Another particular suitable class of O/W emulsifiers are non-ionic self-emulsifying systems derived from olive oil e.g., known as (INCI Name) cetearyl olivate and sorbitan olivate (chemical composition: sorbitan ester and cetearyl ester of olive oil fatty acids) sold under the tradename OLIVEM 1000.

In one particular embodiment, the invention relates to topical composition with all the definitions and preferences given herein in the form of O/W emulsions comprising an oily phase dispersed in an aqueous phase in the presence of an O/W emulsifier wherein the O/W emulsifier is potassium cetyl phosphate. The amount of oily phase in such O/W emulsions is preferably at least 10 wt.-%, more preferably in the range of 10 to 60 wt.-%, most preferably in the range of 15 to 50 wt.-%, such as in the range of 15 to 40 wt-%, based on the total weight of the composition.

Preferably, the topical composition according to the invention further comprise at least one fatty alcohol (co-emulsifier), such as in particular cetyl alcohol, cetearyl alcohol and/or behenyl alcohol. The total amount of one or several fatty alcohols on the topical compositions according to the invention is preferably selected in the range of about 0.1 to 10.0 wt.-%, in particular in the range of about 0.5 to 6.0 wt.-% with respect to the total weight of the topical composition.

Preferably, the topical compositions according to the invention comprise a thickener in particular if the topical composition is in the form of an emulsion to assist in making the consistency of a product suitable. Preferred thickeners are aluminiumsilicates, xanthan gum, hydroxypropylmethylcellulose, hydroxyethylcellulose, polyacrylates such as Carbopole®, (e.g., Carbopole 980, 981, 1382, 2984, 5984) or mixtures thereof. Further preferred thickeners encompass acrylate/C10-30 alkyl acrylate copolymers (such as e.g., Pemulen TR 1, Pemulen TR 2, Carbopol 1328 by. NOVEON) as well as Aristoflex AVC (INCI: Ammonium Acryloyldimethyltaurate/VP Copolymer).

The topical composition according to the present invention advantageously comprise a preservative. When present, the preservative is preferably used in an amount of 0.1 to 2 wt.-%, more preferably in an amount of 0.5 to 1.5 wt.-%, based on the total weight of the composition.

The topical composition according to the invention in general have a pH in the range of 3 to 10, preferably a pH in the range of 4 to 8 and most preferably a pH in the range of 4 to 7.5 such as in the range of 5 to 6.5. The pH can easily be adjusted as desired with suitable acids, such as citric acid or bases, such as sodium hydroxide (e.g., as aqueous solution), triethanolamine (TEA Care), Tromethamine (Trizma Base), and Aminomethyl Propanol (AMP-Ultra PC 2000), according to standard methods in the art.

The amount of the cosmetic composition to be applied to the skin is not critical and can easily be adjusted by a person skilled in the art. Preferably the amount is selected in the range of 0.1 to 3 mg/cm² skin, such as preferably in the range of 0.1 to 2 mg/cm² skin and most preferably in the range of 0.5 to 2 mg/cm² skin.

The following examples are provided to further illustrate the compositions and effects of the present invention. These examples are illustrative only and are not intended to limit the scope of the invention in any way.

### EXAMPLES

### 1. Preparation of Dry Plant Extracts

Dried aerial parts (preferably flowers, stems and/or leaves) of the respective plants are crushed and then extracted with a solvent (mixture of ethanol/water from 50% ethanol/50% water (m/m) to 60% ethanol/40% water) at a temperature selected in the range of RT to 60° C. for one to 3 hours. After filtration the hydro-alcoholic extracts were collected and concentrated under vacuum at a temperature between 5° and 70° C. until obtention of the dry extracts.

### 2. Cultivation of Senescent Cells

Normal and senescent cell populations were obtained from normal human dermal fibroblasts. For the normal cell population, the cells were seeded in cell culture medium (Dulbecco's Modified Eagle Medium supplemented with 10% serum and 1% antibiotics) and allowed to adhere and proliferate in a humidified cell incubator maintaining 37° C. and 5% CO₂. These were then passaged or collected when 90% confluent. For the senescent cell population, the normal fibroblast cells were at this stage shortly exposed to 200 μM H₂O₂ followed by normal cell culture medium. Several days later, the cells were treated again with H₂O₂ to obtain a fully senescent population.

### 3. Senolytic Activity Study

Both normal and senescent populations were mixed into a 70% normal and 30% senescent cell population and represented the treatment population. Afterward, the 70% normal and 30% senescent cell population were seeded in cell culture medium and allowed to adhere in the cell incubator. After adherence, the medium was aspirated and the treatment medium (Dulbecco's Modified Eagle Medium supplemented with 1% serum) containing the respective dry plant extracts at various concentrations (see Table 1) was added to the cells. For controls, there was one condition with treatment medium only without compound (negative control) and one condition with navitoclax, which is a known senolytic (positive control). The cells were incubated in the cell incubator for at least 72 hours. Afterward, senescence-associated beta-galactosidase was visualized—a well-known senescence marker—in the cells to quantify the senescence levels using flow cytometry. The results are depicted in table 1.

### Results

Experimentation showed that the plant extract from Epilobium fleischeri but not from Artemisia umbelliformis, Leontopodium alpinum, Eryngium alpinum and Asparagus officinalis, exhibited a significant senolytic effect and reduced the senescence percentage ratio for human dermal fibroblasts, see Table 1.

TABLE 1

| | | Senescent | Standard | | Significant |
|---|---|---|---|---|---|
| | Concen- | cells | deviation | | senolytic |
| Bioactive | tration | (%) | (%) | P-value | effect |
| CTRL | | 30 | 1 | — | No |
| Navitoclax | 1.5 μM | 10 | 2 | <0.001 | Yes |
| (positive | | | | | |
| control) | | | | | |
| Artemisia | 0.10% | 28 | 1 | 0.068 | No |
| umbelliformis | 0.05% | 28 | 1 | 0.056 | No |
| (Reference) | 0.01% | 29 | 2 | 0.486 | No |
| Leontopodium | 0.001% | 29 | 1 | 0.473 | No |
| alpinum | 0.0005% | 31 | 1 | 0.500 | No |
| (Reference) | 0.0001% | 30 | 1 | 0.951 | No |
| Eryngium | 0.05% | 26 | 2 | <0.05 | Yes |
| alpinum | 0.01% | 28 | 2 | 0.170 | No |
| (Reference) | 0.005% | 32 | 2 | 0.345 | No |
| Asparagus | 0.05% | 39 | 3 | <0.05 | No |
| officinalis | 0.01% | 31 | 2 | 0.467 | No |
| (Reference) | 0.005% | 32 | 3 | 0.198 | No |
| Epilobium | 0.0005% | 9 | 0 | <0.001 | Yes |
| fleischeri | 0.0001% | 11 | 1 | <0.001 | Yes |
| (Invention) | 0.00005% | 16 | 2 | <0.01 | Yes |

Table 1, 70% normal and 30% senescent human dermal fibroblasts were treated for 72 hours with several concentrations of specific plant extracts, which resulted in the reduction of senescent cells for certain plant species. Data are normalized to the untreated control (30% senescent cells and 70% normal cells) and expressed as the mean±SD (n=4).

1. Cosmetic use of an Epilobium fleischeri extract as an active ingredient for the treatment of the signs of aging by selectively eliminating senescent skin cells.

2. The use according to claim 1, wherein the senescent skin cells are human dermal fibroblasts.

3. The use according to claim 1, wherein the senescent cells are age-related senescent cells, stress-related senescent cells, or both.

4. The use according to claim 1, wherein the treatment of the signs of aging is the treatment of fine lines, wrinkles, crow's feet, sagging, skin thinning, age spots as well as the improvement of skin elasticity and skin firmness.

5. The use according to claim 1, wherein the Epilobium fleischeri extract is an aqueous or a dry extract obtained from the flowers, leafs, seeds and/or stems of the plant Epilobium fleischeri.

6. The use according to claim 5, wherein the Epilobium fleischeri extract consists essentially of (i) from 5 to 10 wt.-% based on dry matter of the Epilobium fleischeri flower, leaf and/or stem extract, (ii) from 25 to 50 wt.-% of water, (iii)>50 wt.-% of glycerine, and (iv) from 0.1 to 2 wt.-% of citric acid and/or potassium sorbate.

7. The use according to claim 1, wherein the Epilobium fleischeri extract is administered in the form of a topical composition comprising an effective amount of Epilobium fleischeri extract and a cosmetically acceptable carrier.

8. The use according to claim 7, wherein the carrier consists of at least 30 wt. %, more preferably of at least 40 wt.-%, most preferably of at least 45 wt.-% of water, such as in particular of 50 to 90 wt.-% of water.

9. The use according to claim 7, wherein the topical composition comprises from 0.001 to 10 wt.-%, preferably from 0.01 to 8 wt.-%, even more preferably from 0.1 to 7 wt.-%, furthermore preferably from 0.1 to 5 wt.-% and still more preferably from 0.1 to 3 wt.-%, of the Epilobium fleischeri extract, based on the total weight of the topical composition.

10. The use according to claim 7, wherein the topical composition is a leave-on or rinse-off composition, preferably a leave-on composition.

11. The use according to claim 7, wherein, the composition is an O/W emulsion comprising an oily phase dispersed in an aqueous phase.

12. A cosmetic skin and scalp care method for selective removal of senescent cells in the skin and/or hair follicle, said method encompassing the step of applying, onto the skin or the scalp in need, at least an effective amount of an extract of an Epilobium fleischeri extract; or a composition comprising at least said extract.

13. The method according to claim 12, wherein the senescent cells are senescent human dermal fibroblasts.

14. The method according to claim 12, wherein the effective amount of the Epilobium fleischeri extract (based on dry matter) applied to the skin cells is selected in the range of 0.001 to 1 wt.-%/cm² of skin.

15. The method according to claim 12, wherein the number of senescent cells is reduced by at least 20%, preferably by at least 25%, most preferably by at least 30%.
